(19) United States
(12) Patent Application Publication
(10) Pub. No.: US 2008/0193514 A1
(43) Pub. Date: Aug. 14, 2008

(54) COMPOSTIONS AND METHODS FOR IONTOPHORESIS DELIVERY OF ACTIVE INGREDIENTS THROUGH HAIR FOLLICLES

(75) Inventors: Kentaro Kogure, Kyoto (JP); Masahiko Yamamoto, Sapporo (JP); Hideyoshi Harashima, Sapporo (JP)

Correspondence Address: SEED INTELLECTUAL PROPERTY LAW GROUP PLLC, 701 FIFTH AVE, SUITE 5400, SEATTLE, WA, 98104, US

(73) Assignees: TRANSCU LTD., Singapore (SG); HOKKAIDO UNIVERSITY, Sapporo (JP)

(21) Appl. No.: 11/929,451

(22) Filed: Oct. 30, 2007

Related U.S. Application Data
(60) Provisional application No. 60/886,228, filed on Jan. 23, 2007.

(30) Foreign Application Priority Data
Nov. 2, 2006 (JP) 2006-299448

(51) Int. Cl. A61K 9/127

(52) U.S. Cl. 424/450

(57) ABSTRACT

Systems, devices, and methods for delivering one or more active ingredients to deep regions of hair follicles and intradermal tissues in the vicinity of hair follicles. In some embodiments, a composition is provided including an active ingredient carried in a liposome. The liposome includes a cationic lipid and an amphiphilic glycerophospholipid having a saturated fatty acid moiety and unsaturated fatty acid moiety.

### CROSS-REFERENCES TO RELATED APPLICATIONS

This application claims benefit of U.S. Provisional Application No. 60/886,228 filed Jan. 23, 2007, the contents of which are incorporated herein by reference. This application also claims benefit of priority under 35 U.S.C. § 119 to Japanese Patent Application No. 2006-299448, filed Nov. 2, 2006.

### BACKGROUND

1. Technical Field

This disclosure generally relates to the field of intradermal or transdermal administering of active ingredients by iontophoresis and, more particularly, to compositions useful for delivering active ingredients to deep regions of hair follicles or intradermal tissues in the vicinity of hair follicles by iontophoresis.

2. Description of the Related Art

Iontophoresis employs an electromotive force and/or current to transfer an active agent (e.g., a charged substance, an ionized compound, an ionic a drug, a therapeutic, a bioactive-agent, and the like), to a biological interface (e.g., skin, mucus membrane, and the like), by applying an electrical potential to an electrode proximate an iontophoretic chamber comprising a similarly charged active agent and/or its vehicle. For example, a positively charged ion is transferred into the skin at an anode side of an electric system of an iontophoresis device. In contrast, a negatively charged ion is transferred into the skin at a cathode side of the electric system of the iontophoresis device.

Although skin is one of the most extensive and readily accessible organs, it has historically been difficult to deliver certain active agents transdermally. Often a drug is administered to a living body mainly through the corneum of the skin. The corneum, however, is a lipid-soluble high-density layer that makes the transdermal administration of high water-soluble substances and polymers such as peptides, nucleic acids, and the like difficult.

Commercial acceptance of transdermal delivery devices or pharmaceutically acceptable carriers is dependent on a variety of factors including cost to manufacture, shelf life, stability during storage, efficiency and/or timeliness of active agent delivery, biological capability, and/or disposal issues. Commercial acceptance of transdermal delivery devices or pharmaceutically acceptable carriers is also dependent on their versatility and ease-of-use.

The present disclosure is directed to overcoming one or more of the shortcomings set forth above, and/or providing further related advantages.

### BRIEF SUMMARY

In one aspect, the present disclosure is directed to a composition for administering an active ingredient, through a hair follicle, to a living body by iontophoresis. The composition includes a plurality of liposomes and an active ingredient carried by the liposome. The liposomes may include a cationic lipid and an amphiphilic glycerophospholipid. In some embodiments, the amphiphilic glycerophospholipid comprises a saturated fatty acid moiety and an unsaturated fatty acid moiety. In some embodiments, the liposomes comprise an average liposome diameter ranging from about 400 to about 1000 nm.

In another aspect, the present disclosure is directed to a method for iontophoretically administering one or more active ingredients to deep regions of hair follicles and intradermal tissues in the vicinity of hair follicles by iontophoresis. The method includes providing a composition comprising a plurality of liposomes comprising a cationic lipid, an amphiphilic glycerophospholipid, and the one or more active ingredients. In some embodiments, the amphiphilic glycerophospholipid includes a saturated fatty acid moiety and an unsaturated fatty acid moiety, and the cationic lipid is present in a molar ratio of the cationic lipid to the amphiphilic glycerophospholipid of about 3:7 to about 7:3. The method may further include iontophoretically administering the composition to a living body by iontophoresis using a current ranging from about 0.1 mA/cm² to about 0.6 mA/cm².

In another aspect, the present disclosure is directed to a method of iontophoretically delivering an active ingredient to deep regions of hair follicles and/or intradermal tissues in the vicinity of hair follicles. The method includes enclosing an active ingredient in a liposome with a specific structure for applying the liposome via iontophoresis.

In yet another aspect, the present disclosure is directed to a composition capable of stably and efficiently delivering an active ingredient such as a drug to deep regions of hair follicles and/or intradermal tissues in the vicinity of hair follicles by iontophoresis.

### BRIEF DESCRIPTION OF THE SEVERAL VIEWS OF THE DRAWINGS

In the drawings, identical reference numbers identify similar elements or acts. The sizes and relative positions of elements in the drawings are not necessarily drawn to scale. For example, the shapes of various elements and angles are not drawn to scale, and some of these elements are arbitrarily enlarged and positioned to improve drawing legibility. Further, the particular shapes of the elements as drawn, are not intended to convey any information regarding the actual shape of the particular elements, and have been solely selected for ease of recognition in the drawings.

FIG. 1 is a schematic diagram of an in vitro skin penetration test including an iontophoresis device according to one illustrated embodiment.

FIG. 2 shows a CLSM photograph (A) of a water-soluble fluorochrome (Rhodamine) and a fluorescence labeled NBD (4-chloro-7-nitrobenzofrazan) in a liposome outer layer which was administered to the rat skin in vitro, in a dark field of the skin piece, and a CLSM photograph (B) of a bright field of the same skin piece as that of photograph (A) according to multiple illustrated embodiments.

FIG. 3 shows a CLSM photograph (A) of a fluorescence of Sulfo rhodamine B in an inner layer in the liposome which was administered to rat skin in vitro, and a CLSM photograph (B) of a bright field of the same skin piece as that of photograph (A) according to multiple illustrated embodiments.

FIG. 4 shows a CLSM photograph (A) of a fluorescence of Sulfo rhodamine B which was administered to rat skin in vitro, and a CLSM photograph (B) of a bright field of the same skin piece as that of photograph (A) according to multiple illustrated embodiments.

FIG. 5 is a schematic diagram of the iontophoresis device for performing an in vivo skin penetration test according to one illustrated embodiment.

FIG. 6 shows a CLSM photograph (A) of the fluorescence of Sulfo rhodamine B in the inner layer in the liposome which was administered to rat skin in vivo, and a CLSM photograph (B) of a bright field of the same skin piece as that of photograph (A) according to multiple illustrated embodiments.

FIG. 7 shows a CLSM photograph (A) of the fluorescence of Sulfo rhodamine B which was administered to rat skin in vivo, and a CLSM photograph (B) of a bright field of the same skin piece as that of photograph (A) according to multiple illustrated embodiments.

### DETAILED DESCRIPTION

Unless otherwise specified, the variable “Cn” in a group or as part of a group generally refers to the “total number of carbon atoms n” in the group or the part of a group. Thus, for example, “C₁₋₆ saturated fatty acid” refers to a “saturated fatty acid containing from 1 to 6 carbon atoms”, and “C₁₂₋₃₁ cholesteryl fatty acid ester” refers to a “cholesteryl fatty acid ester containing from 12 to 31 carbon atoms”.

The terms “alkyl”, “alkenyl”, or “alkynyl” as a group or as part of a group generally refer to, unless otherwise specified, straight chain, branched chain, cyclic, substituted, or unsubstituted hydrocarbon radicals. In some embodiments, the “alkyl”, “alkenyl”, or “alkynyl” are selected from the group consisting of straight chain alkyls, alkenyls, or alkynyls and branched chain alkyls, alkenyls, or alkynyls. In some embodiments, the “alkyl”, “alkenyl”, or “alkynyl” is selected from the group consisting of straight chain alkyls, alkenyls, and alkynyls.

The term “aryl” generally refers to, unless otherwise specified, aromatic monocyclic or multicyclic hydrocarbon ring system consisting only of hydrogen and carbon and containing from 6 to 19 carbon atoms, where the ring system may be partially or fully saturated. Aryl groups include, but are not limited to, groups such as phenyl and naphthyl.

The term “heteroaryl” generally refers to, unless otherwise specified, a 5- to 6-membered partially or fully aromatic ring radical which consists of one to three heteroatoms selected from the group consisting of nitrogen, oxygen, and sulfur.

The term “front surface” generally refers to a side near the skin of a living body on the path of electric current flowing through the inside of the electrode structure in administering liposomes.

The term “living body” generally includes mammals such as, for example, human, rats, guinea pigs, rabbits, mice, dogs, cats, and pigs.

Iontophoresis delivery of active ingredients may provide a way of avoiding the first-pass effect of the liver, and may permit for easier control of initiation, cessation, etc., associated with the administration of a drug.

Although it may be possible to transdermally administer substances with various physico-chemical properties using charged liposomes as a carriers (see e.g., Median V M et al., International Journal of Pharmaceutics, Dec. 8, 2005:306(1-2):1-14. Epub Nov. 2, 2005 Epub 2005 Nov. 2), the large particle diameter of liposomes, often make it difficult to pass through the corneum.

Hair follicles, which are connected from the skin surface to a deep region of the skin, may provide a route of transdermally administering liposomes efficiently (e.g., Hoffman R T et al., Nat. Med. 1995 July; 1(7):705-706; Fleisher D et al, Life Sci. 1995; 57 (13):1293-1297). It may be possible to, for example, administer liposomes enclosing an enzyme to hair follicle stem cells in hair follicles by iontophoresis (see e.g., Protopapa E E et al., J Eur Acad Dermatol Venereol. 1999 July; 13(1):28-35). It may also be possible to, for example, administer liposomes enclosing 5-aminolevulinic acid serving as an agent for a photodynamic therapy to the hair follicle sebaceous gland and the like in upper regions of hair follicles by iontophoresis (see e.g., Han I et al., Arch Dermatol Res. 2005 November; 295(5):210-217. Epub 2005 Nov. 11). Han I et al. has also reported that liposomes enclosing adriamycin serving as an agent for treating hair follicle-associated tumors may be delivered to hair follicles by iontophoresis (Han I et al., Exp Dermatol. 2004 February; 13(2):86-92).

Often in iontophoresis, a drug is administered to upper regions of skin tissues. In some embodiments, a drug is systemically administered to a general circulation system through subcutaneous blood vessels that often exist in deep regions of hair follicles. In embodiments where antibody production inducement is intended while targeting, for example, Langerhans' cells and the like (which exist in intradermal tissues in the vicinity of hair follicles), a drug such as a vaccine may be delivered to the intradermal tissues in the vicinity of hair follicles. An object of iontophoresis targeting hair follicles is to stably and efficiently deliver liposomes enclosing a drug to deep regions of hair follicles and intradermal tissues in the vicinity of hair follicles.

### Composition for Iontophoresis

As described above, in some embodiments, the disclosed composition includes an active ingredient carried in a liposome, in which the liposome includes, as a constituent component, a cationic lipid, and an amphiphilic glycerophospholipid including both saturated fatty acid and an unsaturated fatty acid moieties. It is an unexpected fact that liposomes comprising such specific constituent components advantageously provide stable deliver of an active ingredient to deep regions of hair follicles and/or intradermal tissues in the vicinity of hair follicles by iontophoresis.

In some embodiments, a composition is provided for administering an active ingredient through a hair follicle to a living body by iontophoresis. The composition includes a plurality of liposomes and an active ingredient carried by the liposomes. The liposomes may include a cationic lipid and an amphiphilic glycerophospholipid.

The cationic lipid may comprise a C₁₋₂₀ alkane substituted with a C₁₋₂₀ acyloxy group and a triC₁₋₄ alkylammonium group. In some embodiments, the C₁₋₂₀ alkane is a C₁₋₅ alkane. In some other embodiments, the C₁₋₂₀ alkane is a C₁₋₃ alkane. In some embodiments, the C₁₋₂₀ alkane may comprise from one to four C₁₋₂₀ acyloxy groups. In some embodiments, the C₁₋₂₀ alkane may comprise two C₁₋₂₀ acyloxy groups. In some embodiments, the C₁₋₂₂ acyloxy groups are C₁₋₂₀ acyloxy groups. In some embodiments, the C₁₋₂₂ acyloxy groups are C₁₋₁₈ acyloxy groups.

In addition, specific examples of the C₁-C₂₂ acyloxy group may include an alkyl carbonyloxy group, an akenyl carbonyloxy group, an alkynyl carbonyloxy group, an aryl carbonyloxy group, or a heteroaryl carbonyloxy group. In some embodiments, the C₁-C₂₂ acyloxy group is selected from the group consisting of an alkyl carbonyloxy group, an akenyl carbonyloxy group, and an alkynyl carbonyloxy. In some embodiments, the C₁-C₂₂ acyloxy group is an akenyl carbonyloxy group.

The above-mentioned C₁₋₂₀ alkane may include, as a substituent, preferably one to four triC₁₋₆ alkylammonium groups. In some embodiments, the C₁₋₂₀ alkane may include one triC₁₋₆ alkylammonium group. In some embodiments, the triC₁₋₆ alkylammonium groups are triC₁₋₄ alkylammonium groups. In some embodiments, the triC₁₋₆ alkylammonium groups may carry one or more counter ions. Examples of counter ions of the above-mentioned trialkylammonium group include, but not limited to, chlorine ions, bromine ions, iodine ions, fluorine ions, sulfurous ions, nitrous ions, etc. In some embodiments, the counter ion is a chlorine ion, bromine ion, or iodine ion.

Specific examples of the cationic lipid include preferably 1,2-dioleoyloxy-3-trimethylammonium propane (DOTAP), dioctadecyldimethylammonium chloride (DODAC), N-(2,3-dioleyloxy)propyl-N,N,N-trimethylammonium (DOTMA), didodecylammonium bromide (DDAB), 1,2-dimyristoyloxypropyl-3-dimethylhydroxyethylammonium (DMRIE), and 2,3-dioleyloxy-N-[2(sperminecarboxamido)ethyl]-N,N,-dimethyl-1-propanaminum trifluoroacetate (DOSPA). In some embodiments, the cationic lipid is DOTAP.

In some embodiments, the amphiphilic glycerophospholipid comprises a saturated fatty acid moiety and an unsaturated fatty acid moiety.

In some embodiments, the amphiphilic glycerophospholipid includes both a saturated fatty acid and an unsaturated fatty acid as a constituent fatty acid. In some embodiments, the amphiphilic glycerophospholipid is selected from the group consisting of phosphatidylcholine, phosphatidylethanolamine, phosphatidylglycerol, phosphatidic acid, cardiolipin, phosphatidylserine, phosphatidylinositol, and the like. In some embodiments, the amphiphilic glycerophospholipid is phosphatidylcholine. In some embodiments, the amphiphilic glycerophospholipid is an egg-yolk phosphatidylcholine.

In some embodiments, the amphiphilic glycerophospholipid includes a saturated fatty acid selected from the group consisting of C₁₂₋₂₂ saturated fatty acids and C₁₄₋₁₈ saturated fatty acids. In some embodiments, the amphiphilic glycerophospholipid comprises at least one fatty acid selected from the group consisting of palmitic acid, lauric acid, myristic acid, pentadecylic acid, margaric acid, stearic acid, tuberculostearic acid, arachidic acid, and behenic acid. In some embodiments, the amphiphilic glycerophospholipid comprises at least one fatty acid selected from the group consisting of palmitic acid, myristic acid, pentadecylic acid, margaric acid, and stearic acid.

Among the unsaturated fatty acid moieties, examples include C₁₄₋₂₂ unsaturated fatty acids and C₁₄₋₂₀ unsaturated fatty acids. In some embodiments, the unsaturated fatty acid comprises from 1 to 6 carbon-carbon double bonds. In some embodiments, the unsaturated fatty acid comprises from 1 to 4 carbon-carbon double bonds.

In some embodiments, the unsaturated fatty acid includes at least one moiety selected from the group consisting of oleic acid, myristoleic acid, palmitoleic acid, elaidic acid, vaccenic acid, gadoleic acid, ercic acid, nervonic acid, linoleic acid, α-linoleic acid, eleostearic acid, stearidonic acid, arachidonic acid, eicosapentaenoic acid, clupanodonic acid, and docosahexaenoic acid. In some embodiments, the unsaturated fatty acid includes at least one moiety selected from the group consisting of oleic acid, myristoleic acid, palmitoleic acid, elaidic acid, vaccenic acid, gadoleic acid, ercic acid, nervonic acid, linoleic acid, α-linoleic acid, eleostearic acid, stearidonic acid, and arachidonic acid.

In some embodiments, the amphiphilic glycerophospholipid includes both a saturated fatty acid moiety and a unsaturated fatty acid moiety. The saturated fatty acid moiety includes at least one moiety selected from the group consisting of palmitic acid, myristic acid, pentadecylic acid, margaric acid, and stearic acid, and the unsaturated fatty acid moiety includes at least one moiety selected from the group consisting of oleic acid, myristoleic acid, palmitoleic acid, elaidic acid, vaccenic acid, gadoleic acid, ercic acid, nervonic acid, linoleic acid, α-linoleic acid, eleostearic acid, stearidonic acid, and arachidonic acid.

In some embodiments, the liposomes further comprise a sterol as a constituent component. The sterol may be selected from the group consisting of cholesterol, C₁₂₋₃₁ cholesteryl fatty acid, C₁₂₋₃₁ dihydrocholesteryl fatty acid, polyoxyethylene cholesteryl ether, and polyoxyethylene dihydrocholesteryl ether. In some embodiments, the sterol may be selected from the group consisting of cholesterol, cholesteryl lanolate, cholesteryl oleate, cholesteryl nonanate, cholesteryl macadaminate, and dihydrocholesterol polyethylene glycol ether (specifically, DIHYDROCHOLETH-30 is mentioned). In some embodiments, the sterol is Cholesterol.

In some embodiments, the fatty acid such as, for example, cholesteryl fatty acid, dihydrocholesteryl fatty acid, and the like may be saturated or unsaturated. In some embodiments, the fatty acid may be a straight chain, branched chain, or cyclic fatty acid. In some embodiments, the fatty acid moiety in the cholesteryl fatty acid may be a straight chain fatty acid, and the fatty acid moiety in the dihydrocholesteryl fatty acid may be a straight chain fatty acid.

The liposomes may comprise an active ingredient, a cationic lipid, and an amphiphilic glycerophospholipid. The stability and iontophoretic delivery efficiency of the liposomes may depend on the ratio of the cationic lipid to the amphiphilic glycerophospholipid present in the liposomes. In some embodiments, a molar ratio of the cationic lipid to the amphiphilic glycerophospholipid ranges from about 3:7 to about 7:3. In some embodiments, a molar ratio of the cationic lipid to the amphiphilic glycerophospholipid ranges from about 4:6 to about 6:4. In some embodiments, when the liposomes include a sterol, a molar ratio of the cationic lipid to the sterol ranges from about 3:7 to about 7:3. In some embodiments, a molar ratio of the cationic lipid to the sterol ranges from about 4:6 to about 6:4.

In some embodiments, a molar ratio of the amphiphilic glycerophospholipid to the sterol ranges from about 3:7 to about 7:3. In some embodiments, a molar ratio of the amphiphilic glycerophospholipid to the sterol ranges from about 4:6 to about 6:4. In some embodiments, a molar ratio of the cationic lipid to the total of the amphiphilic glycerophospholipid and the sterol ranges from about 3:7 to about 7:3. In some embodiments, a molar ratio of the cationic lipid to the total of the amphiphilic glycerophospholipid and the sterol ranges from about 4:6 to about 6:4. In some embodiments, a molar ratio of the cationic lipid, to the amphiphilic glycerophospholipid, and to the sterol is about 2:1:1.

In some embodiments, the average particle diameter of the liposomes is about 400 nm or greater. In some embodiments, the average particle diameter of the liposomes ranges from about 400 nm to about 1000 nm. The average particle diameter of the liposomes can be confirmed by, for example, a dynamic-light-scattering method, a static-light-scattering method, an electron microscope observation method, and an atomic force microscope observation method.

The active ingredient may comprise a hydrophobic substance or a water soluble substance and may comprise a non-charged substance or a charged substance insofar as it can be carried (e.g., enclosed) in liposome. Examples of active ingredients capable of being carried in a liposome include low molecular weight compounds and high molecular weight compounds (e.g., nucleic acids, peptides, etc.). Further examples of active ingredients include drugs (e.g., vaccines, hair-growth agents, hair restorers, hair removers, hormones, etc.), colorants, nucleic acids (e.g., DNA, RNA, PNA, etc.), peptides, proteins, enzymes, lipopolysaccharides, cell components, etc. Examples of cell components include cell wall fractions, fibrous structure fractions, pilus component fractions, glucosyl transferase (GTF) fractions, and protein antigen fractions, or any cell component that can be used as an antigen. The amount of active ingredient enclosed in the liposome can be suitably determined in view of physico-chemical properties, doses, etc., of the active ingredient.

The disclosed liposomes and composition comprising the liposomes may be prepared in a variety of ways. In some embodiments, the disclosed liposomes and liposome compositions may be prepared by the following Example 1.

### EXAMPLE 1

First, cationic lipid, amphiphilic glycerophospholipid, and, as required, sterol or the like are mixed in desired ratios in an organic solvent such as CHCl₃ to obtain a suspension. The suspension is distilled under reduced pressure, and the addition of an organic solvent and distillation under reduced pressure are repeated, to yield a lipid film. Next, to the lipid film, a buffer such as 10 to 50 mM HEPES (2-[4-(2-hydroxyethy)-1 piperazinyl]ethanesulfonic acid) or the like and a desired amount of active ingredient are added. The obtained mixed liquid is left standing at room temperature for 10 minutes for hydration, followed by sonication. The sonication is performed in a sonicator, for example, at room temperature at 85 W for 1 minute, but the conditions are not limited thereto. The mixed liquid is treated using a membrane filter, extruder, etc., to adjust the particle diameter, thereby obtaining liposomes. The liposomes are further mixed with a pharmacologically acceptable carrier and the like, thereby obtaining a composition of liposomes.

A number of pharmacologically acceptable carriers and excipients may be used with the disclosed compositions and methods insofar as the administration of liposomes by iontophoresis is not substantially hindered. For example, surfactants, lubricants, dispersants, buffers such as HEPES, additives such as preservatives, solubilizing agents, antiseptics, stabilizing agents, antioxidants, colorants, may be included. The liposome composition can be formed into a suitable dosage form as desired, insofar as the administration of liposomes by iontophoresis is not substantially hindered.

In some embodiments, the composition of liposomes is formed into a solution or suspension with HEPES buffer and/or any of the disclosed electrolytes. The disclosed composition and methods can be applied to various uses according to types and properties of an active ingredient to be enclosed in liposome. In some embodiments, when a drug is used as the active ingredient, the disclosed composition can be used as medicine. Therefore, in some embodiments, the disclosed liposomes can be used for producing pharmaceutical compositions. In some embodiments, the liposome compositions may be stably or efficiently delivered to deep regions and/or intradermal tissues of hair follicles, and may be used for localized delivery of intradermal vaccines. In some embodiments, the liposomes compositions may be used to administer active agents, provide treatment, and the like of various diseases or conditions requiring a systemic or localized delivery of active ingredients.

In some embodiments, a method of administering an active ingredient to a living body by iontophoresis includes placing any of the disclosed compositions on the skin surface of a living body, and applying an electric current to the skin. In some embodiments, the active ingredients are enclosed in the liposomes in the composition and administered to a living body through hair follicles.

In some embodiments, the disclosed composition may be directly placed on the skin surface, or may be part of an electrode structure of an iontophoresis device in which the composition is held, stored, or carried. In use, electric current is applied to an electrode structure holding, storing, or carrying a composition of liposomes enclosing the active ingredient, and administered iontophoretically.

For cationic liposomes, the anode of an iontophoresis is supplied with an electric current. In some embodiments, the electric current supplied by the iontophoretic device and applied to the liposomes ranges from about 0.1 to about 0.6 mA/cm². In some embodiments, the electric current supplied by the iontophoretic device ranges from about 0.3 mA/cm² to about 0.5 mA/cm². In some embodiments, the electric current supplied by the iontophoretic device is about 0.45 A/cm². In some embodiments, a period of time for applying electric current to the electrode structure ranges from about 0.5 hours to about 1.5 hours, in some embodiments, from about 0.75 hours to about 1.25 hours, and, in some further embodiments, about 1 hour.

### Electrode Structure and Device for Iontophoresis

In some embodiments, the disclosed compositions and/or liposomes may be held in, stored, carried, or be part of, an electrode structure suitable for iontophoretic delivery of the compositions and/or liposomes. In some embodiments, the electrode structure for administering an active ingredient to a living body by iontophoresis comprises one or more of the disclosed compositions. In some embodiments, the liposomes take the form of cationic liposomes, and the electrode structure is configured such that the anode side of the electrode structure is configured to transdermally deliver the composition including the liposomes, when current and/or a potential is applied to the electrode structure.

In some embodiments, the electrode structure includes at least a positive electrode and an active ingredient holding unit capable of holding any of the disclosed compositions or liposomes. In some embodiments, the active ingredient holding unit may be directly disposed on the front surface of the positive electrode and other components such as an ion exchange membrane, may be disposed between the positive electrode and the active ingredient holding unit insofar as the administration of liposomes by iontophoresis is not substantially hindered. In some embodiments, the electrode structure comprises at least a positive electrode, an electrolyte holding unit for holding electrolyte disposed on the front surface of the positive electrode, an anion exchange membrane disposed on the front surface of the electrolyte holding unit, and an active ingredient holding unit for holding any of the disclosed compositions or liposomes. In some embodiments, on the front surface of the above-mentioned active ingredient holding unit, a cation exchange membrane may be disposed as desired.

In some embodiments, an iontophoresis device may include any of the disclosed electrode structures, or any other structure suitable for iontophoretic delivery of the active ingredient. In some embodiments, the iontophoresis device may include at least a power unit, an electrode structure connected to the power unit and holding any of the disclosed compositions or liposomes, and an electrode structure as a counter electrode of the electrode structure. The structure of the electrode structure as a counter electrode is not limited insofar as the administration of liposomes by iontophoresis is not substantially hindered. For example, the electrode structure as a counter electrode may include a negative electrode, an electrolyte holding unit for holding electrolyte disposed on the front surface of the negative electrode, and an ion exchange membrane disposed on the front surface of the electrolyte holding unit. The above-mentioned ion exchange membrane may be an anion exchange membrane or a cation exchange membrane, and preferable is an anion exchange membrane.

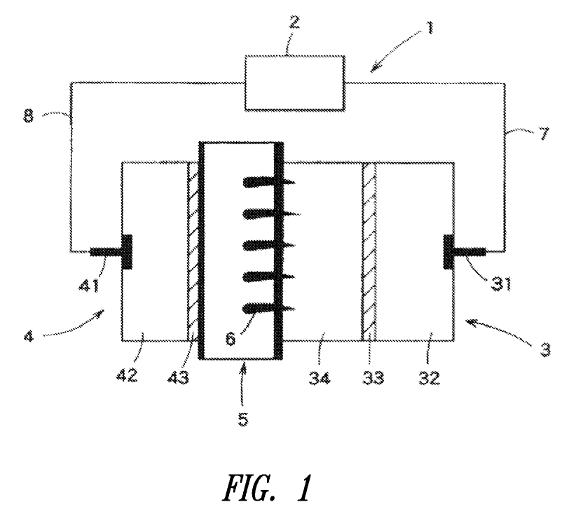

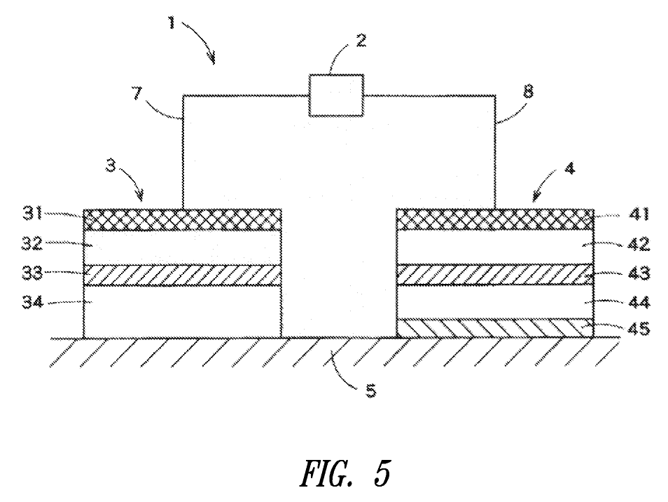

Examples of an electrode structure and an iontophoresis device are illustrated in FIGS. 1 and 5 and include those disclosed in International Publication WO 03/037425 A1.

Liposomes may migrate to a side opposite to the positive electrode due to an electric field (electric field) resulting from applying an electric current, and may be efficiently emitted from the electrode structure. In some embodiments, a method of operating an iontophoresis device, includes disposing the electrode structure comprising a plurality of liposomes carrying an active ingredient, and the counter electrode structure, on the skin surface of a living body, and applying a sufficient electric current to the iontophoresis device, so as to emit a substantial amount of the liposomes held in active ingredient holding unit of the electrode structure.

In the above-mentioned iontophoresis device, the active ingredient holding unit or the electrolyte holding unit may be formed of a reservoir (electrode chamber) which is, for example, formed of acryl and is filled with any of the disclosed compositions or liposomes, or with an electrolyte and may be formed of a thin film body having properties of holding the disclosed compositions or liposomes, or electrolyte. With respect to the thin film body, the same material can be used in the active ingredient holding unit and the electrolyte holding unit.

As the electrolyte, a desired electrolyte can be suitably used according to conditions of the active ingredient to be applied. However, electrolytes that adversely affect the skin of a living body due to electrode reaction should be avoided. Suitable electrolytes include organic acid and salts thereof which exist in a metabolic cycle of a living body are preferable from the viewpoint of non-toxicity. For example, lactic acid and fumaric acid are preferable and, specifically, an aqueous solution in which a ratio of 1M lactic acid to 1M sodium fumarate is 1:1 is preferable.

It is important for the thin film body forming the active ingredient holding unit to have sufficient ability to absorb and retain a composition and electrolyte and to have sufficient ability to migrate ionized liposomes impregnated in and/or retained by the thin film body under predetermined electric field conditions to the skin side (ion transportation ability, ion electrical conductivity). As a material having both favorable absorbance and retaining properties and favorable ion transportation ability, an acrylic resin hydrogel substance (acrylic hydrogel film), a segmented polyurethane gel film, an ion electorical-conductive porous sheet for forming a gel solid electrolyte (e.g., porous polymer disclosed in JP 11-273452 A which includes an acryl-nitrile copolymer, as a base, having acrylonitrile in a proportion of 50 mol % or more, and preferably 70 to 98 mol % and having a porosity of 20 to 80%), or the like is mentioned. When impregnating the above-mentioned active ingredient holding unit, the impregnation degree (100×(WD)/D[%], where D represents a dry weight and W represents a weight after impregnation) is preferably 30 to 40%.

The conditions for impregnating the composition of the present or electrolyte into the active ingredient holding unit or the electrolyte holding unit are suitably determined according to the impregnation amount of electrolyte and an ionic drug, the impregnation rate, etc. The impregnation is performed, for example, at 40° C. for 30 minutes.

As an electrode of the electrode structure, an inert electrode comprising, for example, an electrically conductive material such as carbon and platinum is preferably used.

As the ion exchange membrane used for the electrode structure, it is preferable to use a cation exchange membrane and an anion exchange membrane in combination. As the cation exchange membrane, NEOSEPTA CM-1, CM-2, CMX, CMS, CMB, and CLE04-2 manufactured by Tokuyama Corporation, and the like are preferably mentioned. As the anion exchange membrane, NEOSEPTA AM-1, AM-3, AMX, AHA, ACH, ACS, ALE04-2, and AIP-21 manufactured by Tokuyama Corporation, and the like are preferably mentioned. In some embodiments, the cation exchange membrane comprises a porous film including an ion exchange resin (having cation exchange functionality) impregnated and/or distributed in a portion or within the pores of the porous film. In some embodiments, the anion exchange membrane comprises an ion exchange resin having an anion exchange functionality.

Details of each of the above-mentioned constituent material are disclosed in International Publication WO 03/037425A1 filed commonly owned by applicants, and incorporated by reference herein.

### EXAMPLES

### Test Example 1

### EXAMINATION OF CONDITIONS OF APPLYING ELECTRIC CURRENT IN DELIVERING LIPOSOMES THROUGH HAIR FOLLICLE

### Preparation of Liposomes

350 μL of CHCl₃ solution of 10 mM DOTAP (Avanti Polar Lipids, Inc.), 150 μL of CHCl₃ solution of 10 mM cholesterol (hereinafter referred to as “Chol”, Avanti Polar Lipids, Inc.), and 6.4 μL of CHCl₃ solution of Rho-DOPE (1,2-dioleoyl-sn-glycero-3-phosphoethanolamine-N (lissamine rhodamine B sulfonyl)) were mixed. 500 μL of CHCl₃ was added to the mixture, thereby obtaining a suspension (molar ratio; DOTAP:Chol:Rho-DOPE=7:3:0.1). The suspension was distilled under reduced pressure using an evaporator, and then 400 μL of CHCl₃ was added, followed by distillation under reduced pressure again, thereby obtaining a lipid film. 1 mL of 10 mM HEPES buffer was added to the lipid film. The obtained mixed liquid was left standing at room temperature for 10 minutes for hydration, and then sonication (AU-25C ultrasonic cleaner, product of Aiwa Ika kogyo k.k.) was performed at room temperature at 85 W for 1 minute. Further, the mixed liquid was treated using a PC membrane with a pore size of 400 nm and a PC membrane with a pore size of 100 nm (product name: Nuclepre Track-Etch Membrane, product of Whatman) by an extruder (product name: Mini-Extruder, product of Avanti Polar Lipids, Inc.), thereby obtaining a liposome suspension.

### Test of Applying Electric Current

The hair on the back of a SD rat (male, 10-weeks old, CLEA Japan, Inc.) was shaved, and the skin was harvested. Next, 100 μL of the above-obtained liposome suspension was applied to the surface of the skin. Next, as illustrated in FIG. 1, an iontophoresis device 1 equipped with a power unit 2, a working electrode structure 3, and a non-working electrode structure 4 as a counter electrode was disposed on skin 5. Here, the working electrode structure 3 was disposed on the front surface side of the skin 5 having hair follicles 6; the non-working electrode structure 4 as a counter electrode was disposed on the rear surface side of the skin 5; and both the electrode structures 3 and 4 were connected to the power unit through cords 7 and 8, respectively. The working electrode structure 3 included a positive electrode 31, an electrolyte holding unit 32 holding 1 mL of electrolyte, the unit which was disposed on the front surface of the positive electrode 31, an anion exchange membrane 33, and an active ingredient holding unit 34 holding 850 μL of liposome suspension. The non-working electrode structure 4 included a negative electrode 41, an electrolyte holding unit 42 holding 1 mL of electrolyte, the unit which was disposed adjacent to the negative electrode 41, and a cation exchange membrane 43.

The active ingredient holding unit 34 and the electrolyte holding unit (32, 42) employed an acrylic reservoir capable of retaining the active ingredient or the electrolyte in the interior space thereof. The above-mentioned anion exchange membrane 33 (product name: ALE04-2, product of Tokuyama Corporation) and the above-mentioned cation exchange membrane 43 (product name: CLE04-2, product of Tokuyama Corporation) were kept in physiological saline prior to use. The electrolyte was electrolyte comprising disodium fumarate (420 mM), L-ascorbic acid 2-trisodium phosphate (18.5 mM), and polyacrylic acid (0.4 mM).

Next, electric current was applied to the iontophoresis device 1 illustrated in FIG. 1 under various conditions indicated in Table 1 shown below. After the application of electric current, the skin 5 was removed from the iontophoresis device and the surface was wiped off with a filter paper. Then, the skin piece was collected and embedded in an OTC compound using liquid nitrogen. Further, a 15-μm-thick piece was cut from the obtained frozen block with a cryostat (CM3000, product of Leica), and fluorescence of rhodamine in the cut piece was observed under a confocal laser scanning microscope (CLSM).

The conditions of applying electric current to the iontophoresis device 1 and the test results are shown in Table 1.

In the following table 1, ++ refers to a condition in which the delivery of liposomes was observed in a deep region by 50% or more with respect to the length of a hair follicle, + refers to a condition in which the delivery of liposomes was observed in a deep region within the range of 0 (hair follicle entrance) to less than 50% with respect to the length of a hair follicle, and − refers to a condition in which existence of liposomes was not confirmed in hair follicles.

TABLE 1

| | 1 hr | 2 hr | 3 hr |
|---|---|---|---|
| 0.94 mA (0.3 mA/cm²) | − | + | ++ |
| 1.41 mA (0.45 mA/cm²) | ++ | ++ | − |
| 1.88 mA (0.6 mA/cm²) | − | − | − |

In view of the results shown in Table 1, electric current was applied at 1.41 mA (0.45 mA/cm²) for 1 hour in the following tests.

### Test Example 2

### EXAMINATION OF COMPOSITION OF LIPID LIPOSOME

### Preparation of Liposomes

Liposomes of a lipid composition (molar ratio) shown in the following Table 2 was prepared. In the following, “EPC” refers to egg-yolk phosphatidylcholine (product of Nippon Yushi, Co., Ltd.), “DOPE” refers to 1,2-dioleoyl-sn-glycero-3-phosphoethanolamine (product of Avanti Polar Lipids, Inc.), “CHEMS” refers to Cholesteryl hemisuccinate (product of Avanti Polar Lipids, Inc.), and “DOTAP” and “Chol” are as mentioned above.

TABLE 2
Cationic liposome (lipid composition; molar ratio)

| | |
|---|---|
| (a) | DOTAP/Chol = 7/3 |
| (b) | DOTAP/Chol = 5/5 |
| (c) | DOTAP/EPC = 5/5 |
| (d) | DOTAP/EPC = 3/7 |
| (e) | DOTAP/DOPE = 5/5 |
| (f) | DOTA/EPC/Chol = 5/2.5/2.5 |
| (g) | DOTAP/EPC/Chol = 5/4/1 |
| (h) | DOTAP/EPC/DOPE = 5/2.5/2.5 |

Anionic liposome (lipid composition; molar ratio)

| | |
|---|---|
| (i) | CHEMS/EPC = 2/9 |
| (j) | CHEMS/DOPE = 2/9 |

In the preparation of liposome, each lipid was mixed so that the molar ratio was as shown in Table 2, and 1 mol % Rhodamine-DOPE was added as a label. Then, liposomes were obtained in the same manner as in Test Example 1. For example, in Table 2(b), 350 μL of CHCl₃ solution of 10 mM DOTAP and CHCl₃ solution of 10 mM Chol were mixed. Further, 1 mol % Rhodamine-DOPE (6.4 μL) was added as a label, and then liposomes were obtained in the same manner as in Test Example 1.

### Iontophoresis Test

Using liposomes shown in Table 2, an iontophoresis test was performed under conditions of applying electric current at 1.41 mA (0.45 mA/cm²) for 1 hour. When using cationic liposomes shown in (a) to (h), Table 2, the iontophoresis device 1 illustrated in FIG. 1 was used. In contrast, when using anionic liposomes shown in (i) and (j), Table 2, an iontophoresis device setup included a negative electrode and a cation exchange membrane disposed in place of the positive electrode 31 and the anion exchange membrane 33 of the working electrode structure 3, and a positive electrode and an anion exchange membrane disposed in place of the negative electrode 41 and the cation exchange membrane 43 of the non-working electrode structure 4.

The results were as shown in Table 3. In Table 3, + refers to a condition in which the delivery of liposomes was observed in a deep region within the range of 0 (hair follicle entrance) to less than 50% with respect to the length of a hair follicle, ++ refers to a condition in which the delivery of liposomes was observed in a deep region by 50 to 75% with respect to the length of a hair follicle, +++ refers to a condition in which the delivery of liposomes was observed in a deep region by 76 to 100% or more with respect to the length of a hair follicle, and—refers to a condition in which existence of liposomes was not confirmed in hair follicles.

TABLE 3

| | Lipid composition | | | | | | | | | |
|---|---|---|---|---|---|---|---|---|---|---|
| | (a) | (b) | (c) | (d) | (e) | (f) | (g) | (h) | (i) | (j) |
| Impregnation ability | + | − | ++ | + | − | +++ | ++ | + | − | + |
| into hair follicles | | | | | | | | | | |

As shown in Table 3, (c) comprising DOTAP and EPC and (f) and (g) each comprising DOTAP, EPC, and Chol showed ++ and +++.

### Test Example 3

### EXAMINATION OF INFLUENCE OF PARTICLE DIAMETER OF LIPOSOME ON DLIVERY THROUGH HAIR FOLLICLE

Preparation of Liposomes with Various Particle Diameters

Liposomes (DOTA/EPC/Chol=5/2.5/2.5) having the same composition as that of (f) of Table 2 were prepared following the same procedure as in Test Example 1. Next, in accordance with a procedure described below, the particle size of the liposomes was adjusted.

### 3-a: Particle Diameter About 400 nm

After the mixed liquid was left standing at room temperature for 10 minutes for hydration in Test Example 1, the obtained lipid film was treated with a VORTEX (Tube mixer TRIO HM-2F, product of Asone) for 30 seconds, thereby preparing a liposome suspension with an average particle diameter of about 400 nm.

### 3-b: Particle Diameter About 250 nm

Following the procedure of 3-a, the mixed liquid was treated with an extruder using a 400-nm PC membrane, thereby obtaining a liposome suspension with an average particle diameter of about 200 nm. Then, the suspension was subjected to freezing treatment for 30 seconds using liquid nitrogen and subjected to melting treatment at 40° C. for 3 minutes, and this cycle was repeated three times, thereby preparing a liposome suspension with an average particle diameter of about 250 nm. The average particle diameter was confirmed by a dynamic-light-scattering method (Photal ELS-8000HO, product of Otsuka electronics).

### 3-c: Particle Diameter About 200 nm

The liposome suspension obtained in 3-a was treated with an extruder using a 400-nm PC membrane, thereby obtaining a liposome suspension with an average particle diameter of about 200 nm.

### 3-d: Particle Diameter About 150 nm

Following the procedure of Test Example 1 except for not treating with an extruder, a liposome suspension with an average particle diameter of about 150 nm was prepared.

### 3-e: Particle Diameter About 100 nm

Following the procedure of Test Example 1, a liposome suspension with an average particle diameter of about 100 nm was prepared.

### Iontophoresis Test

Using the liposome suspensions 3-a to 3-e, iontophoresis was performed under the same conditions as those of Test Example 2.

The results were as shown in Table 4. In Table 4, + and +++ are similarly defined as in Table 3.

TABLE 4

| | Average Particle Diameter (nm) | | | | |
|---|---|---|---|---|---|
| | 100 | 150 | 200 | 250 | 400 |
| Impregnation ability into hair follicles | + | + | + | + | +++ |

The liposomes with an average particle diameter of about 400 nm were delivered to deep regions of hair follicles, such as Bulge region. In contrast, the liposomes with particle diameters of about 250 to 100 nm were not delivered to deep regions of hair follicles, such as Bulge region.

### Test Example 4

### CONFIRMATION TEST OF DELIVERY OF LIPOSOME IN VITRO

### Preparation of Liposome

Liposomes having an outer layer labeled with NBD (4-chloro-7-nitrobenzofrazan) and an inner layer labeled with Sulfo rhodamine B was prepared by the following procedure.

250 μL of CHCl₃ solution of 10 mM DOTAP, 125 μL of CHCl₃ solution of 10 mM EPC, 125 μL of CHCl₃ solution of 10 mM Chol, and 9.2 μL of CHCl₃ solution of NBD-DOPE were mixed. 500 μL of CHCl₃ was added to the mixture, thereby obtaining a suspension (DOTAP:EPC:Chol: NBD-DOPE=7:3:0.1). The suspension was distilled under reduced pressure using an evaporator, and then 400 μL of CHCl₃ was added, followed by distillation under reduced pressure again, thereby obtaining a lipid film. 1 mL of HEPES buffer solution of 2.5 mM Sulfo rhodamine was added to the lipid film. The obtained mixed liquid was left standing at room temperature for 10 minutes for hydration, and then sonication (AU-25C ultrasonic cleaner, product of Aiwa Ika kogyo k.k.) was performed at room temperature at 85 W for 1 minute. Further, the mixed liquid was treated using a PC membrane with a pore size of 400 nm and a PC membrane with a pore size of 100 nm (product name: Nuclepre Track-Etch Membrane, product of Whatman) by an extruder (product name: Mini-Extruder, product of Avanti Polar Lipids, Inc.), thereby obtaining a suspension. The suspension was further subjected to ultracentrifugation at 20° C. at 5300 rpm for 4 hours, and separated Sulfo rhodamine was removed.

### Iontophoresis Test

Iontophoresis was performed using the above-obtained liposome suspension under the same conditions as those of Test Example 3. As control, an HEPES buffer solution of 2.5 mM Sulfo rhodamine was used. A photograph (×10) was taken with a confocal laser scanning microscope (CLSM; LSM510 META, product of Zeiss) using wavelengths λex=475 nm and λem=540 nm in detection of NBD labeling the outer layer of the liposome and using wavelengths λex=570 nm and λem=590 nm in detection of Sulfo rhodamine B.

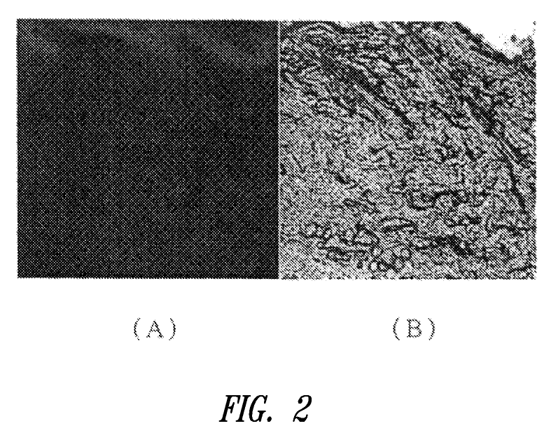

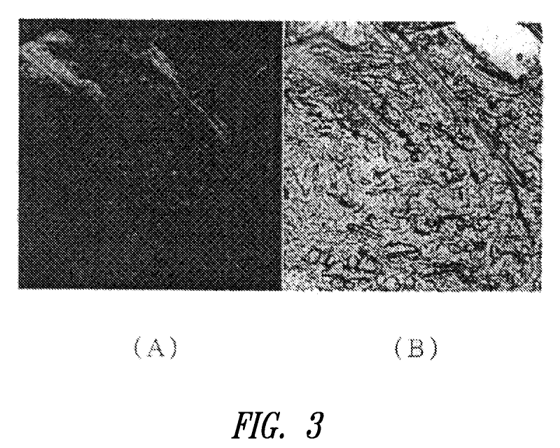

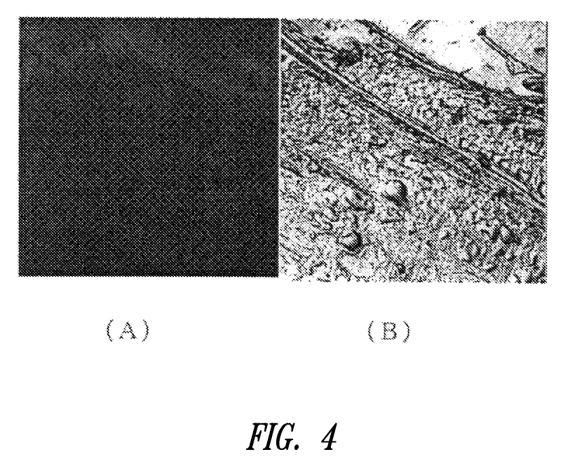

The results were as shown in FIGS. 2 to 4. In FIGS. 2 to 4, (A) is a photograph, taken with a fluorescence microscope, of a water-soluble fluorochrome (Rhodamine) and a fluorescence labeled lipid (NBD) in a dark field of the skin piece and (B) is a photograph, taken with the microscope, of a bright field of the same skin piece as that of (A). When the liposome suspension was used, NBD (FIG. 2) labeling the outer layer of the liposome and Sulfo rhodamine B (FIG. 3) labeling the inner layer thereof were detected at the same portion, and it was confirmed that the liposomes were delivered into hair follicles with their structures being retained. In contrast, Sulfo rhodamine B (FIG. 4) as control was not detected in hair follicles, and it was confirmed that Sulfo rhodamine B was not delivered into hair follicles as it was.

### Test Example 5

### CONFIRMATION TEST OF DELIVERY OF LIPOSOME THROUGH HAIR FOLLICLE

### Preparation of Liposome

A liposome suspension was prepared in the same manner as in Test Example 4.

Iontophoresis Test using SD Rat

Anesthesia (ketamine/xylazine=10/1, 1 mg per weight kg) was administered to an SD rat (male, 10-weeks old, CLEA Japan, Inc.), and the hair on the back was shaved. Next, the iontophoresis device 1 comprising the power unit 2, the working electrode structure 3, and the non-working electrode structure 4 was disposed on the exposed skin 5 as illustrated in FIG. 5. Here, 100 μL of the above-mentioned liposome suspension was applied beforehand to the contact surface of the exposed skin 5 and the working electrode structure 3. In the above-mentioned iontophoresis device 1, the working electrode structure 3 had the same structure as that of Test Example 1, and, more particularly, the working electrode structure 3 had the positive electrode 31, the electrolyte holding unit 32 holding 1 mL of electrolyte disposed on the front surface of the positive electrode 31, the anion exchange membrane 33, and the active ingredient holding unit 34 holding 850 μL of liposome suspension disposed on the front surface of the anion exchange membrane 33. In contrast, the non-working electrode structure 4 had the negative electrode 41, the electrolyte holding unit 42 retaining 1 mL of electrolyte disposed on the front surface of the negative electrode 41, the cation exchange membrane 43, an electrolyte holding unit 44 holding 800 μL of physiological saline disposed on the front surface of the cation exchange membrane 43, and an anion exchange membrane 45 disposed on the front surface of the electrolyte holding unit 44. The above-mentioned anion exchange membranes 33 and 45 (ALE04-2, product of Tokuyama Corporation) and the cation exchange membrane 43 (CLE04-2, product of Tokuyama Corporation) were kept in physiological saline beforehand for use.

Next, an iontophoresis test was performed under conditions of applying electric current at 1.41 mA (0.45 mA/cm²) for 1 hour. 3 hours after the completion of applying electric current, the SD rat was euthanized with carbon dioxide gas. Then, the skin 5 where the first electrode structure 3 was disposed was harvested, and a skin piece was obtained in the same manner as in Test Example 1. With respect to the skin piece, NBD labeling the outer layer of liposome and Sulfo rhodamine B were detected in the same manner as in Test Example 4.

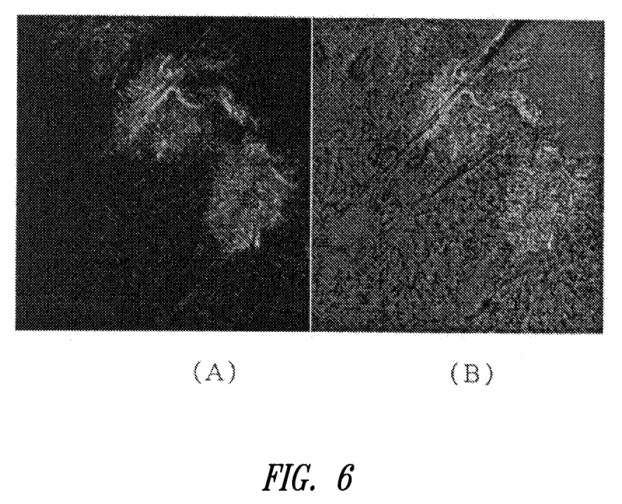

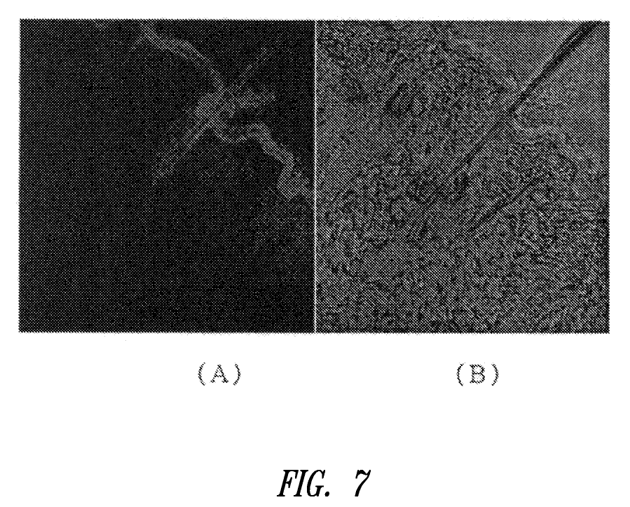

The results were as shown in FIGS. 6 and 7. In FIGS. 6 and 7, (A) is a photograph, taken with a fluorescence microscope, of a water-soluble fluorochrome (Rhodamine) and a fluorescence labeled lipid (NBD) in a dark field of the skin piece and (B) is a photograph, taken with the microscope, of a bright field of the same skin piece as that of (A). Also in vivo, the fluorescence of NBD (FIG. 6) and the fluorescence of Sulfo rhodamine B (FIG. 7) were detected at the same portion. In vivo test, it was confirmed that the liposomes were diffused from hair follicles to intradermal tissues with the structure being retained.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

What is claimed is:

1. A composition for administering an active ingredient through a hair follicle to a living body by iontophoresis, comprising:
a plurality of liposomes comprising:
a cationic lipid, and
an amphiphilic glycerophospholipid, the amphiphilic glycerophospholipid having a saturated fatty acid moiety and an unsaturated fatty acid moiety; and
an active ingredient carried by the liposome.

2. The composition according to claim 1 wherein the cationic lipid is a C₁₋₂₀ alkane substituted with a C₁₋₂₂ acyloxy group and a triC₁₋₆ alkylammonium group.

3. The composition according to claim 1 wherein the cationic lipid comprises two C₁₋₂₂ acyloxy groups and one triC₁₋₆ alkylammonium group.

4. The composition according to claim 1 wherein the cationic lipid is 1,2-dioleoyloxy-3-(trimethylammonium)propane.

5. The composition according to claim 1 wherein the amphiphilic glycerophospholipid is phosphatidylcholine or an egg-yolk phosphatidylcholine.

6. The composition according to claim 1, wherein the saturated fatty acid moiety is a C₁₂₋₂₂ saturated fatty acid.

7. The composition according to claim 1 wherein the unsaturated fatty acid moiety is selected from the group consisting of palmitic acid, lauric acid, myristic acid, pentadecanoic acid, margaric acid, stearic acid, tuberculostearic acid, arachidic acid, and behenic acid.

8. The composition according to claim 1 wherein the unsaturated fatty acid moiety comprises 1, 2, 3, 4, 5 or 6 carbon-carbon unsaturated double bonds.

9. The composition according to claim 1 wherein the unsaturated fatty acid moiety is C₁₄₋₂₂ unsaturated fatty acid.

10. The composition according to claim 1 wherein the unsaturated fatty acid moiety is selected from the group consisting of oleic acid, myristoleic acid, palmitoleic acid, elaidic acid, vaccenic acid, gadoleic acid, erucic acid, nervonic acid, linolic acid, α-linoleic acid, eleostearic acid, stearidonic acid, arachidonic acid, eicosapentaenoic acid, clupanodonic acid, and docosahexaenoic acid.

11. The composition according to claim 1, wherein a molar ratio of the cationic lipid to the amphiphilic glycerophospholipid is from about 3:7 to about 7:3.

12. The composition according to claim 1, wherein the liposome further comprises a sterol, the sterol present in a molar ratio of the cationic lipid to the sterol of from about 3:7 to about 7:3.

13. The composition according to claim 12 wherein the sterol is selected from the group consisting of cholesterol, C₁₂₋₃₁ cholesteryl fatty acid, C₁₂₋₃₁ dihydrocholesteryl fatty acid, polyoxyethylene cholesteryl ether, and polyoxyethylene dihydrocholesteryl ether.

14. The composition according to claim 12 wherein the sterol is selected from the group consisting of cholesterol, cholesteryl lanolate, cholesteryl oleate, cholesteryl nonanate, cholesteryl macadaminate, and polyoxyethylene dihydrocholesteryl ether.

15. The composition according to claim 12 wherein the sterol is cholesterol.

16. The composition according to claim 12 wherein a molar ratio of the amphiphilic glycerophospholipid to the sterol is from about 3:7 to about 7:3.

17. The composition according to claim 12 wherein a molar ratio of the cationic lipid to the total of the amphiphilic glycerophospholipid and the sterol is from about 3:7 to about 7:3.

18. The composition according to claim 12 wherein a molar ratio of the cationic lipid, to the amphiphilic glycerophospholipid, and to the sterol is about 2:1:1.

19. The composition according to claim 1 wherein an average particle diameter of the liposome is about 400 nm or more.

20. The composition according to claim 1 wherein an average particle diameter of the liposome ranges from about 400 nm to about 1000 nm.

21. The composition according to claim 1 wherein the active ingredient is selected from the group consisting of a drug, a colorant, a nucleic acid, a protein, an enzyme, a peptide, a lipopolysaccharide, and a cell component.

22. A method for iontophoretically administering one or more active ingredients to deep regions of hair follicles and/or intradermal tissues in the vicinity of hair follicles by iontophoresis, comprising:
providing a composition comprising a plurality of liposomes having an average liposome diameter ranging from about 400 to about 1000 nm, the plurality of liposomes comprising a cationic lipid, an amphiphilic glycerophospholipid, and the one or more active ingredients, the amphiphilic glycerophospholipid having a saturated fatty acid moiety and an unsaturated fatty acid moiety, and the cationic lipid present in a molar ratio of the cationic lipid to the amphiphilic glycerophospholipid of about 3:7 to about 7:3; and
iontophoretically administering the composition to a living body by iontophoresis using a current ranging from about 0.1 mA/cm² to about 0.6 mA/cm².

23. The method according to claim 22, wherein providing a composition comprising the plurality of liposomes includes providing the amphiphilic glycerophospholipid having a C₁₂₋₂₂ saturated fatty acid moiety and a moiety is C₁₄₋₂₂ unsaturated fatty acid moiety.

24. The method according to claim 22, wherein the plurality of liposomes further comprise a sterol; and wherein providing a composition comprising the plurality of liposomes includes providing the plurality of liposomes having the sterol present in a molar ratio of the cationic lipid to the sterol of about 3:7 to about 7:3.

25. The method according to claim 22, wherein the plurality of liposomes further comprise a sterol selected from the group consisting of cholesterol, C₁₂₋₃₁ cholesteryl fatty acid, C₁₂₋₃₁ dihydrocholesteryl fatty acid, polyoxyethylene cholesteryl ether, and polyoxyethylene dihydrocholesteryl ether; and
wherein providing a composition comprising the plurality of liposomes includes providing the plurality of liposomes including the sterol present in a molar ratio of the cationic lipid to the sterol of about 3:7 to about 7:3.
